(19) United States
(12) Patent Application Publication
(10) Pub. No.: US 2026/0196328 A1
(43) Pub. Date: Jul. 9, 2026

(54) INTELLIGENT RECOVERY APPLICATION

(71) Applicant: Therabody, Inc., Los Angeles, CA (US)

(72) Inventors: Timothy ROBERTS, Sarasota, FL (US); Caitlin BERZOK, New York, NY (US); Jason MORRIS, Sewickley, PA (US); Noah WEISEL, Los Angeles, CA (US); Daniel HAYRAPETIAN, Santa Monica, CA (US)

(21) Appl. No.: 19/439,143

(22) Filed: Jan. 2, 2026

Related U.S. Application Data
(60) Provisional application No. 63/741,688, filed on Jan. 3, 2025.

(51) Int. Cl. G16H 20/30; A61H 23/00

(52) U.S. Cl. CPC G16H 20/30; A61H 23/006

(57) ABSTRACT

An electronic computing device can receive user information. The user information includes usage history of a therapeutic device received through electronic communication from the therapeutic device. The electronic computing device can also generate a prompt including the user information, instructions to select a user activity based on the user information, instructions to output a recommendation of the user activity, and instructions to output the recommendation in a standardized data format. The electronic computing device can send the prompt to an artificial intelligence model and receive an output including the recommendation from the artificial intelligence model.

### CROSS-REFERENCE TO RELATED APPLICATIONS

This application claims the benefit of U.S. Provisional Application No. 63/741,688, filed on January 3, 2025, the entirety of which is incorporated herein by reference.

### BACKGROUND

Individual health and fitness goals vary substantially. Beyond that, each person differs in respects that affect how any fitness goal could be achieved. Coaches can evaluate their clients’ circumstances and goals and write appropriate training programs, but many people lack the inclination or means to work with a coach. Even someone working with a coach may benefit from additional guidance, such as when the coach provides a training program but little guidance on recovery.

Recently, artificial intelligence models have become capable of simulating reasonable competence in a number of fields. However, a user may encounter some difficulties in working with artificial intelligence models. For example, artificial intelligence models can hallucinate, and an uninformed user may struggle to distinguish a model’s useful output from its hallucinations. Artificial intelligence models may also struggle to arrange the information they output in a manner that matches the utility and visual appeal that purpose-built applications can provide for predetermined information.

Digital fitness applications exist, but they fall far short of the kind of instruction a coach could provide. For example, fitness applications may be used to track a user’s exercise, such as by recording distances run or counting steps, but current fitness applications may lack the capability to tell a user how to reach specific health or fitness goals.

### SUMMARY

Accordingly, there may be a need for a way to integrate the capabilities of artificial intelligence into a purpose-built fitness application environment. Aspects of the present application relate to an application configured to act as an interface between a user and an artificial intelligence model. The application may be configured to gather relevant user information, such as the user’s characteristics, recent activities, and goals, and to instruct an artificial intelligence model to recommend activities in furtherance of the user’s goals. The application may be configured to constrain the artificial intelligence model’s recommendations, such as by instructing the artificial intelligence model to recommend recovery protocols from a library of predefined activities, thereby preventing the artificial intelligence model from making inappropriate recommendations. In some embodiments, the predefined activities may involve usage of a device in communication with the application, such as a percussive massage device, so that the application may send parameters for the recommended activities to the device or may automatically detect completion of the activities from information received from the device. The application may further be configured to instruct the artificial intelligence model to output its recommendations in a standardized data format so that the application may handle the recommendations easily and present them in a useful manner.

Some aspects of the present disclosure relate to an electronic computing device configured to receive user information. The user information may include usage history of a therapeutic device received through electronic communication from the therapeutic device. The electronic computing device may be configured to generate a prompt including the user information, instructions to select a user activity based on the user information, instructions to output a recommendation of the user activity, and instructions to output the recommendation in a standardized data format. The electronic computing device may also be configured to send the prompt to an artificial intelligence model. The electronic computing device may also be configured to receive an output comprising the recommendation from the artificial intelligence model. The electronic computing device may also be configured to derive a deliverable from the output by processing the output in a routine configured to generate deliverables from information in the standardized data format. The electronic computing device may also be configured to display the deliverable.

In some embodiments according to the foregoing, the instructions to select the user activity may include instructions to select the user activity from a predefined group of activities.

In some embodiments according to any of the foregoing, the electronic computing device may further be configured to display the deliverable within a graphical user interface. The deliverable may be derived from the output.

In some embodiments according to any of the foregoing, the routine is configured to arrange information in the standardized data format in a graphical display format.

In some embodiments according to any of the foregoing, the electronic computing device may further be configured to receive user feedback on the recommendation and send the feedback to the artificial intelligence model.

In some embodiments according to any of the foregoing, the user activity may include a therapeutic activity.

In some embodiments according to any of the foregoing, the user information may include a user characteristic.

In some embodiments according to any of the foregoing, the user information may include activity history.

In some embodiments according to any of the foregoing, the activity history may include information transmitted from an activity monitoring device.

In some embodiments according to any of the foregoing, the user information may include a user goal.

In some embodiments according to any of the foregoing, the electronic computing device may be configured to detect completion of the user activity from data transmitted from the therapeutic device.

Some aspects of the present disclosure relate to an electronic computing device configured to receive user information. The user information may include user goals. The user information may also include activity history transmitted from an activity monitoring device. The electronic computing device may also be configured to prompt an artificial intelligence model to select a recovery activity based on the user information and to output a recommendation comprising the recovery activity. The electronic computing device may also be configured to display a deliverable derived from the recommendation.

In some embodiments according to the foregoing, the recommendation may be one among a plurality of recommendations included by the deliverable. Each recommendation among the plurality of recommendations may include a time aspect. The electronic computing device may be configured to display the deliverable in a format that lists the plurality of recommendations in chronological order according to the time aspect of each recommendation.

In some embodiments according to any of the foregoing, the electronic computing device may further be configured to detect completion of the recovery activity from data transmitted from a therapeutic device.

Some aspects of the present disclosure relate to a method of generating and providing instructions to a user. Providing instructions to a user may also be referred to as prompting the user, such as prompting the user to engage in certain activity. Prompting a user may take the form of coaching the user, though prompting a user as described herein is not limited to that purpose. Prompts to the user are distinct from prompts to an artificial intelligence model within the context of the present disclosure. Prompts to a user may be referred to as “user prompts” and prompts to an artificial intelligence model may be referred to as “model prompts.” The method may include requesting user information from a user. The method may also include generating a model prompt including the user information. The model prompt may also include instructions to select a user activity based on the user information. The model prompt may also include instructions to output a recommendation of the user activity. The model prompt may also include instructions to output the recommendation in a standardized data format. The method may also include sending the model prompt to an artificial intelligence model. The method may also include generating a deliverable by processing an output comprising the recommendation in a routine configured to generate deliverables from information in the standardized data format. The method may also include providing the deliverable to the user.

In some embodiments according to the foregoing, the method may also include receiving user feedback on the recommendation and training the artificial intelligence model on the user feedback.

In some embodiments according to any of the foregoing, the method may also include tracking completions of recommended activities by the user. The recommended activities may include the user activity. The method may also include providing feedback to the user based on the completions.

In some embodiments according to any of the foregoing, the user activity may include usage of a therapeutic device. Tracking completions of recommended activities may include receiving data transmitted by the therapeutic device.

In some embodiments according to any of the foregoing, the method may also include building a predetermined group of user activities. The model prompt may include instructions to select the user activity from the predetermined group of user activities.

In some embodiments according to any of the foregoing, receiving the user data comprises receiving data transmitted from an activity monitoring device.

Further features and advantages, as well as the structure and operation of various embodiments, are described in detail below with reference to the accompanying drawings. It is noted that the specific embodiments described herein are not intended to be limiting. Such embodiments are presented herein for illustrative purposes only. Additional embodiments will be apparent to persons skilled in the relevant art(s) based on the teachings contained herein.

### BRIEF DESCRIPTION OF THE DRAWINGS

The accompanying drawings, which are incorporated herein and form a part of the specification, illustrate embodiments of the present disclosure and, together with the description, further serve to explain the principles of the disclosure and to enable a person skilled in the pertinent art to make and use the disclosure.

FIG. 1 schematically illustrates an intelligent recovery system according to some aspects of the present application.

FIG. 2 is a flowchart of an intelligent user prompt generation process according to some aspects of the present application.

FIG. 3 is a flowchart of a setup workflow for an intelligent user prompt generating application according to some aspects of the present application.

FIG. 4 is a flowchart of an artificial intelligence workflow for an intelligent user prompt generating application according to some aspects of the present application.

FIG. 5 illustrates a screen of a graphical user interface of an intelligent user prompt generating application according to some aspects of the present application.

Embodiments of the present disclosure will be described with reference to the accompanying drawings.

### DETAILED DESCRIPTION

The following Detailed Description refers to accompanying drawings to illustrate exemplary embodiments consistent with the disclosure. References in the Detailed Description to "one exemplary embodiment," "an exemplary embodiment," "an example exemplary embodiment," etc., indicate that the exemplary embodiment described may include a particular feature, structure, or characteristic, but every exemplary embodiment might not necessarily include the particular feature, structure, or characteristic. Moreover, such phrases are not necessarily referring to the same exemplary embodiment. Further, when a particular feature, structure, or characteristic is described in connection with an exemplary embodiment, it is within the knowledge of those skilled in the relevant art(s) to affect such feature, structure, or characteristic in connection with other exemplary embodiments whether or not explicitly described.

The exemplary embodiments described herein are provided for illustrative purposes, and are not limiting. Other exemplary embodiments are possible, and modifications may be made to the exemplary embodiments within the spirit and scope of the disclosure. Therefore, the Detailed Description is not meant to limit the disclosure. Rather, the scope of the disclosure is defined only in accordance with the following claims and their equivalents.

Embodiments may be implemented in hardware (e.g., circuits), firmware, software, or any combination thereof. Embodiments may also be implemented as instructions stored on a machine-readable medium, which may be read and executed by one or more processors. A machine-readable medium may include any mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others. Further, firmware, software, routines, instructions may be described herein as performing certain actions. However, it should be appreciated that such descriptions are merely for convenience and that such actions in fact result from computing devices, processors, controllers, or other devices executing the firmware, software, routines, instructions, etc. Further, any of the implementation variations may be carried out by a general purpose computer, as described below.

For purposes of this disclosure, the term “module” may include one, or more than one, component within an actual device, and each component that forms a part of the described module may function either cooperatively or independently of any other component forming a part of the module. Conversely, multiple modules described herein may represent a single component within an actual device. Further, components within a module may be in a single device or distributed among multiple devices in a wired or wireless manner.

The following Detailed Description of the exemplary embodiments will so fully reveal the general nature of the disclosure that others can, by applying knowledge of those skilled in relevant art(s), readily modify and/or adapt for various applications such exemplary embodiments, without undue experimentation, without departing from the spirit and scope of the disclosure. Therefore, such adaptations and modifications are intended to be within the meaning and plurality of equivalents of the exemplary embodiments based upon the teaching and guidance presented herein. It is to be understood that the phraseology or terminology herein is for the purpose of description and not of limitation, such that the terminology or phraseology of the present specification is to be interpreted by those skilled in relevant art(s) in light of the teachings herein.

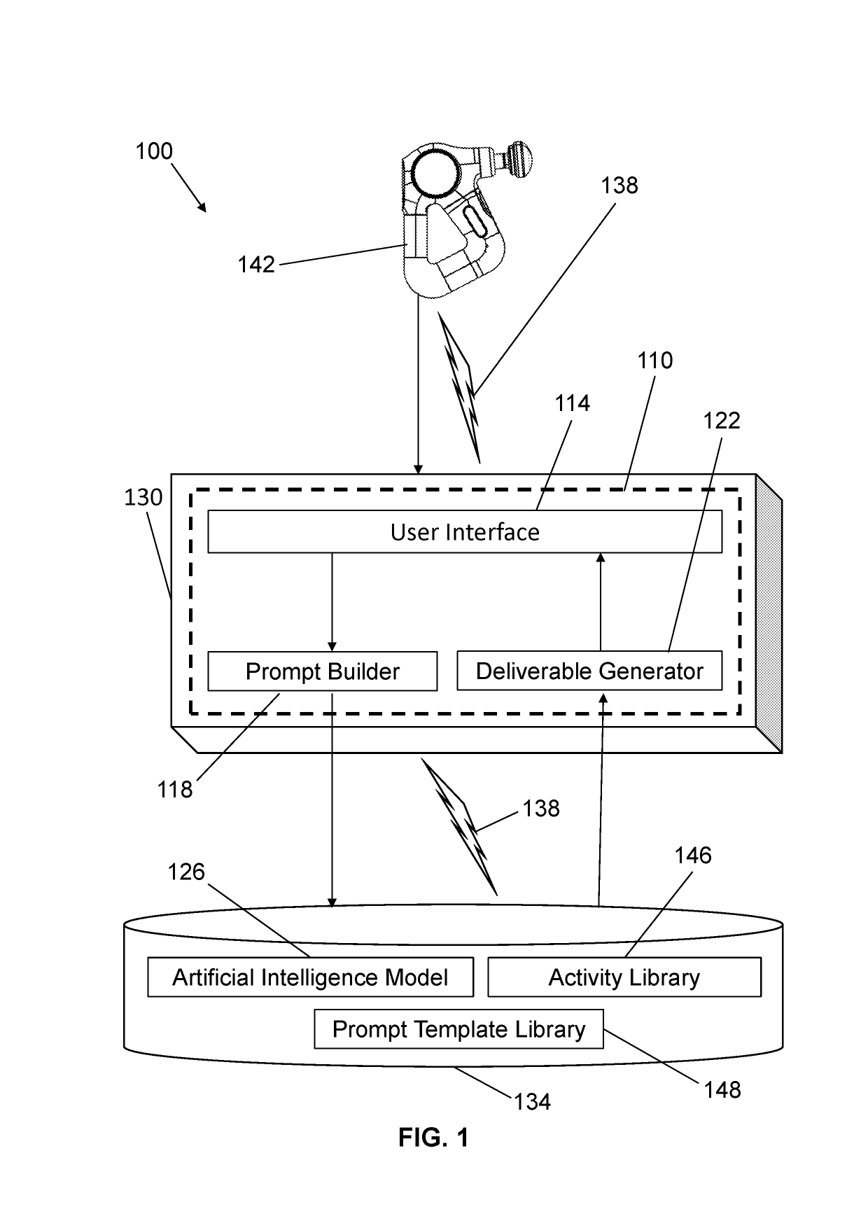

FIG. 1 illustrates a system 100 for generating prompts for a user. Prompts for a user may be referred to as user prompts. Generating user prompts may take the form of providing intelligent recovery coaching to a user, but the systems and methods for generating user prompts described herein are not limited to that purpose. System 100 comprises an application 110 and an artificial intelligence model 126.

Application 110 is configured for receiving user information, building prompts for an artificial intelligence model 126 based on the user information, deriving deliverables from outputs from the artificial intelligence model 126, and displaying the deliverables. Prompts for artificial intelligence model 126 may be referred to as model prompts. Accordingly, application 110 comprises a user interface 114. User interface 114 may comprise, for example, a graphical user interface, or any other digital elements capable of both presenting information to a user and receiving user information from the user.

The user information may comprise any information about a user that may be provided to artificial intelligence model 126 for artificial intelligence model 126 to consider when formulating recovery and musculoskeletal health activity recommendations for the user. Thus, user information may comprise a user’s goals, such as weight loss, muscle gain, cardiovascular endurance, skill at specific sports, or specific athletic metrics such as capacity to run a certain distance, lift a certain weight, or learn a specific athletic skill. User information may also comprise physical or demographic information about a user such as age, height, weight, sex, any injuries or disabilities, any areas of pain, and measures of a user’s current athletic capabilities. User information may also comprise a user’s activity history, such as any recovery activities, fitness activities, activities for treating musculoskeletal health needs, sleep activities, or any combination of the foregoing, a user has performed within a predetermined timeframe. User information may also comprise musculoskeletal health data, such as self-reported or monitored acute and chronic pain, pain location, pain intensity levels, pain type, posture (including static and/or dynamic posture data), movement data, image data, or any combination of the foregoing. User information may also comprise sleep data, such as sleep durations, types of sleep (light sleep, deep sleep, and rapid eye movement (REM) sleep), time spent awake, time to fall asleep, and the like. In further embodiments, user information may also comprise biometric data, such as heart rate, heart rate variability (HRV), skin temperature, blood pressure, EEG readings, ECG readings, blood oxygen, glucose (in blood, sweat, interstitial fluid, etc.), facial data, image data, combinations thereof, and the like.

Artificial intelligence model 126 according to some embodiments may comprise an instanced artificial intelligence model. Here, an instanced artificial intelligence model refers to a type of artificial intelligence model that may exist in multiple instances, wherein all instances share a common foundation of architecture and training, but the instances do not share information from model prompts between each other. Thus, user information submitted to the instance of artificial intelligence model 126 used in system 100 would not be sent to instances of artificial intelligence model 126 used for other purposes. For example, large language models are frequently made available to the public for general purpose use, but artificial intelligence model 126 of system 100 may comprise an instance of a large language model that does not share user information or any other information from model prompts it receives with general purpose instances of the same large language model. By using a separate instance of artificial intelligence model 126, system 100 may limit access to user information provided to application 110 and thereby protect the privacy of any users of application 110. To further protect the privacy of users of application 110, system 100 may be set up so that users may only interact with artificial intelligence model 126 through application 110, and application 110 may limit the types of model prompts users may send to artificial intelligence model 126 and the kind of information from artificial intelligence model’s 126 outputs that may be made visible to a user.

In some embodiments, all instances of application 110 may interact with a single instance of artificial intelligence model 126. Where application 110 limits the possible interactions with artificial intelligence model 126, the connection of multiple instances of application 110 to one instance of artificial intelligence model 126 may enable artificial intelligence model 126 to learn quickly from many users’ feedback without compromising the users’ privacy. However, in further embodiments, each instance of application 110 may interact with a different instance of artificial intelligence model 126, thereby ensuring that no user interacts with an instance of artificial intelligence model 126 that has received another user’s information. In further embodiments, each instance of artificial intelligence model 126 may be configured to end, thereby clearing all received user information, upon predetermined events such as a user closing application 110, artificial intelligence model 126 completing generation and delivery of recommendations responsive to a model prompt, or artificial intelligence model 126 integrating and learning from user feedback.

Application 110 further comprises a model prompt builder 118. Model prompt builder 118 is configured to build model prompts for artificial intelligence model 126 based on user inputs. Accordingly, model prompt builder 118 may be configured to build model prompts comprising the user information. Model prompt builder 118 may further be configured to build model prompts comprising instructions to select a user activity based on the user information and instructions to output a recommendation of the selected user activity. Model prompt builder 118 may therefore act on user inputs by building model prompts for artificial intelligence model 126 that cause artificial intelligence model 126 to recommend activities to the user based on information about the user. In some embodiments, model prompt builder 118 may further be configured to instruct artificial intelligence model 126 to provide additional context for the recommended activity, such as a time of day that artificial intelligence model 126 recommends for performing the activity, or which types of activity devices 142 the user has available. Model prompt builder 118 may further be configured to build model prompts including constraints on artificial intelligence model’s 126 recommendations, such as limitations on how many activities may be recommended during predefined windows of time during a day. Model prompt builder 118 may further be configured to build model prompts including a motivation, perspective, or identity for artificial intelligence model 126 to assume when formulating its recommendations, such as an instruction for artificial intelligence model 126 to assume it is a coach to the user.

Model prompt builder 118 may be configured to build model prompts comprising instructions indicating a type of recommendation for artificial intelligence model 126 to generate. In some embodiments, model prompt builder 118 may utilize a model prompt template from model prompt template library 148 as part of building model prompts. A model prompt template may be organized in a modular fashion with each module being configured to generate a specific output from artificial intelligence module 126.

In various embodiments, a model prompt template may be organized using a plurality of functional modules. Each module may be configured to address a particular aspect of an overall output or recommendation goal for artificial intelligence model 126. Modularizing the model prompt template makes it possible to more readily adapt, update, or replace specific desired functionalities of the output without requiring extensive revisions to other parts of the model prompt template. Moreover, the modularized approach facilitates a more rapid and reliable development process, as each module can be validated, tested, and optimized in isolation before being integrated into the complete instruction set.

An example of a model prompt template may comprise a scheduling module, a scheduling example module, an output formatting module, an activity selector module, and an output rules module. In further embodiments, the model prompt template may further comprise a role module. In further embodiments, model prompt templates may comprise any combination of the foregoing modules, and may therefore lack some of the foregoing modules, or may include additional modules not specifically mentioned herein.

The scheduling module may comprise rules for including recommended times for recommended activities in the output. For example, the scheduling module may comprise instructions such as “You will generate recommendations for the user based on data provided to you about the user. Times of day are defined as ‘Morning,’ ‘Afternoon,’ and ‘Nighttime,’ and you should only recommend two activities per time period of each calendar day.” The scheduling example module may comprise examples of suitable outputs including recommendations regarding when to perform recommended activities.

The output formatting module may comprise rules regarding the format in which artificial intelligence model 126 should format its outputs. For example, the output formatting module may comprise instructions such as “Provide your recommendations in the following JSON Format:” followed by an intended format for the output.

The activity selector module may comprise instructions regarding how to select activities to recommend. For example, the activity selector module may comprise instructions such as “When choosing which activities to recommend, use the activity name, activity description, and the body parts used in the activity to prioritize activities that are similar to activities the user has completed recently, match the user’s wellness goals and preferences, are appropriate for the time of day, and have not been recommended in the same calendar day.”

The output rules module may comprise any additional rules to be applied to artificial intelligence module 126. For example, the output rules module may comprise instructions such as “In the output, use a library ID number to identify each activity. Provide only the JSON formatted output without additional explanation. Do not recommend the same activity twice in a calendar day unless the second recommendation of the activity is appropriate to recover from an activity recommended earlier in the day.”

The role module may comprise instructions regarding a role artificial intelligence module 126 should assume when generating the recommendations, such as “You are a coach recommending routines to help users achieve their goals.” The role module may provide artificial intelligence module 126 with guidance regarding how to select appropriate activities beyond the guidance provided expressly in the activity selector module.

The foregoing language in each module is provided by way of example. The modular nature of the model prompt template allows language for each module to be altered while remaining within a framework that can be expected to continue to function consistently. In addition to templates comprising different combinations of modules, model prompt template library 148 may also comprise multiple variations of each module type, and application 110 may select an appropriate template and an appropriate variation on each module type based on user preferences and settings.

In certain embodiments, each module may include placeholders or designated variable fields for inserting variables such as user data, user input, or other relevant data for further customizing the model prompt template to generate output having a specific format but that is personalized for each user. In some embodiments, additional data may be provided from one or more input sources, such as databases, user interfaces, sensors, third-party services, or other data repositories. These placeholders may be implemented as parameter fields, macros, template tags, or other variable constructs that enable the seamless insertion of context-relevant data at runtime or upon compilation of the model prompt from the model prompt template. By mapping these placeholders to particular data inputs, prompt builder 118 can automatically populate a module’s content with relevant values, parameters, or configurations, eliminating the need for manual data insertion and reducing the potential for human error.

The modularization of the model prompt improves flexibility and scalability of model prompt generation while ensuring that output generated from the model prompt has formatting that is reliable and consistent and that is personalized for each user. For example, one module may handle routine scheduling, another may handle how routines are generated, and yet another may manage output formatting. Moreover, in embodiments with placeholder variables, linking each module to appropriate data sources allows the model prompt to generate outputs that are personalized and relevant based on current information.

Returning to the recommendations, the type of recommendation may be a recommendation of an activity for the user to perform. In some embodiments, model prompt builder 118 may be configured to build model prompts including instructions to artificial intelligence model 126 to generate a recommendation of a recovery activity for a user to perform. By building model prompts including instructions to consider the user information and to output recommendations for activities for a user to perform, model prompt builder 118 may cause artificial intelligence model 126 to output recommendations of activities that are tailored to a user’s goals, physical characteristics, and recent activity. Model prompt builder 118 may, for example, prompt artificial intelligence model 126 to recommend one or more recovery protocols, such as recovery protocols using a therapeutic activity device 142, to recover from the user’s recent athletic activities in a manner suitable for the user’s physical characteristics overall goals.

In some embodiments, system 100 may comprise an activity library 146. As used herein, a library may refer to a digital collection of information. Thus, activity library 146 may comprise a digital collection of predetermined activities that a user may perform. The contents of activity library 146 may be predetermined at the time that any request for recommendations is made to artificial intelligence model 126, but may be periodically updated throughout the lifetime of system 100. In some embodiments, the activities of activity library 146 may comprise therapeutic activities, such as activities for recovering from athletic activity, activities for treating musculoskeletal health needs, and/or sleep activities. In further embodiments, all activities in activity library 146 may be activities for recovering from athletic activity. In further embodiments, the activities of activity library 146 may comprise protocols for using predetermined therapeutic activity devices 142 to recover from athletic activity. In further embodiments, all activities in activity library may be protocols for using predetermined therapeutic activity devices 142 to recover from athletic activity. In further embodiments, activities of activity library 146 may comprise athletic and movement based exercises and protocols for recovering from athletic activity or treating musculoskeletal health needs. In further embodiments, activities of activity library 146 may comprise protocols for using predetermined therapeutic activity devices 142 (e.g., sleep mask, eye massager, or the like) for sleep activities.

Model prompt builder 118 may be configured to generate model prompts for artificial intelligence model 126 that instruct artificial intelligence model 126 to recommend only activities selected from activity library. Model prompt builder 118 may thereby leverage the flexible analytic and predictive capabilities of artificial intelligence model 126 while constraining artificial intelligence model’s 126 recommendations to a curated selection of activities embodied in activity library 146. In some embodiments, all activities in activity library 146 may be human authored activities, which can ensure that all activities in activity library are possible, safe, and effective. Instructing artificial intelligence model 126 to recommend only activities selected from activity library 146 may therefore reduce or eliminate the possibility of artificial intelligence model 126 hallucinating an activity that would be impossible or inadvisable for a human to perform. To further ensure the efficacy of artificial intelligence model’s 126 recommendations, some or all activities in activity library 146 may be authored by professionals trained licensed in areas such as personal training, nutrition, physical therapy, chiropractic, or medicine. Model prompt builder 118 may also be configured to generate model prompts that instruct artificial intelligence model 126 to recommend only activities selected from activity library that satisfy certain additional criteria, such as requiring no activity devices 142 other than activity devices 142 paired to electronic computing device 130 or conforming to user preferences.

In some embodiments, model prompt builder 118 may further be configured to build model prompts for artificial intelligence model 126 comprising instructions to output the recommendation in a standardized data format. Standardized data formats that model prompt builder 118 may instruct artificial intelligence model 126 to use for outputs include, for example, JSON, XML, CSV, or any other standard machine readable format. By including in the model prompt instructions to output the recommendation in a standardized data format, application 110 may cause artificial intelligence model 126 to output recommendations in a format that can be presented to the user in predictable manner through user interface 114.

Application 110 further comprises a deliverable generator 122. Deliverable generator 122 comprises a routine configured to convert outputs from artificial intelligence model 126 to deliverables that may be displayed in user interface 114. In this instance, a routine refers to a process within application 110, such as an algorithm. A deliverable from deliverable generator 122 may comprise an arrangement of information in a format expressible through user interface 114.

In embodiments wherein model prompt builder 118 is configured to build model prompts including instructions to output the recommendation in a standardized data format, deliverable generator 122 may comprise a routine configured to generate deliverables from information stored in the standardized data format. Thus, deliverable generator 122 may comprise an algorithm configured to parse the standardized data format. For example, a model prompt builder 118, according to some embodiments, may build model prompts including instructions to output a recommendation in JSON format. In such embodiments, the output from artificial intelligence model 126 may be in JSON format, and may therefore include actionable information for a user in a format that a human reader would have difficulty interpreting. Application 110 may receive the output from artificial intelligence model 126 and parse the output through deliverable generator 122. The resulting deliverable may comprise the artificial intelligence model’s 126 recommendation in a format that can be readily interpreted by the user. Application 110 may be configured to send deliverables from deliverable generator 122 to user interface 114 to be presented to the user through user interface 114.

By first instructing artificial intelligence model 126 to structure outputs in a machine readable standardized data format, then parsing the outputs through a routine for converting information in the standardized data format to deliverables for user interface 114, application 110, according to some embodiments, may leverage the flexible, analytical, and predictive capabilities of an artificial intelligence model 126 while maintaining control over how the model’s recommendations are presented to the user. For example, some forms of artificial intelligence model 126, such as large language models, can be relatively flexible in the structure of model prompts they receive, and they can be trained to generate more useful responses to model prompts than some non-learning algorithms. Unless instructed otherwise, large language models may output their responses in prose. Thus, the outputs of some varieties of artificial intelligence model may be useful, but without graphical elements, cohesion to any particular aesthetic, or suitability for interpretation at-a-glance. However, outputs from artificial intelligence model 126 in standardized data formats may be converted to deliverables that may be presented in any way a designer of application 110 intends. Instructing artificial intelligence model 126 to use a standardized data format may therefore enable application 110 to present the substance of artificial intelligence model’s 126 outputs in appealing ways, such as alongside suitable graphical elements, in accordance with the visual design of the user interface of a specific application, or in an arrangement that can be interpreted more quickly than a paragraph of text. Application 110, according to such embodiments, may further handle or present artificial intelligence model’s 126 recommendations in formats unique to application 110. Artificial intelligence model 126 may lack the capability to output recommendations directly in a format unique to application 110, but it may be possible to use an algorithm or other non-learning computing process within deliverable generator 122 to convert an output from artificial intelligence model 126 in a standardized data format to a unique format.

System 100 may comprise physical elements in addition to application 110 and artificial intelligence model 126. Accordingly, in some embodiments, system 100 comprises an electronic computing device 130 that runs application 110. System 100 may further comprise a remote computing device 134 that hosts artificial intelligence model 126. Electronic computing device 130 may be in electronic communication 138 with remote computing device 134. Electronic communication 138 can include any kind of wired or wireless electronic communication, such as internet communication, communication over a local network, communication by Bluetooth or Wi-Fi protocols, or communication over any other network or protocol.

In some embodiments, electronic computing device 130 can be a personal device, such as a smart phone, tablet, or a computer. In some such embodiments, remote computing device 134 may be a computer or server. Running application 110 on electronic computing device 130 can enable a fast, responsive experience for a user, while running artificial intelligence model 126 on remote computing device 134 can reduce the storage and processing burden on electronic computing device 130. Running artificial intelligence model 126 on remote computing device 134 can also enable artificial intelligence model 126 to be trained on feedback from multiple users, allowing each user of a different electronic computing device 130 and instance of application 110 to benefit as artificial intelligence model 126 learns from all users. While some benefits are associated with this distribution of application 110 and artificial intelligence model 126 on electronic computing device 130 and remote computing device 134, other distributions are possible in other embodiments. For example, artificial intelligence model 126 could run on the same electronic computing device 130 as application 110 to eliminate the need for remote computing device 134. In other examples, some aspects of application 110, such as model prompt builder 118, deliverable generator 122, or both, may run on a remote computing device 134 instead of electronic computing device 130. Activity library 146 and model prompt template library 148 are illustrated as being stored on remote computing device 134 by way of example. However, in other embodiments, activity library 146, model prompt template library 148, or both may be stored within application 110 on electronic computing device 130 or on activity device 142.

Instructing artificial intelligence model 126 to recommend only activities from activity library 146 and to structure outputs in standardized data formats can limit outputs from artificial intelligence model 126 to certain predefined possibilities in machine-readable arrangements. Such limitations can make artificial intelligence model’s 126 outputs easy for application 110 to handle. Such limitations may further enable rich interactions with artificial intelligence model’s 126 recommendations in application 110. For example, application 110 may be able to embed links to related resources in deliverables derived from artificial intelligence model’s 126 outputs. In a further example, for each activity recommended in an output from artificial intelligence model 126, application 110 may be configured to embed a link to a corresponding page comprising instructions for performing the activity. In some such embodiments, the deliverable may comprise displaying a text name or icon for each recommended activity, and a user may be able to navigate to a corresponding instruction page for each recommended activity by tapping or clicking on the activity’s name or icon in the deliverable.

In some embodiments, system 100 may further comprise an activity device 142 in electronic communication 138 with electronic computing device 130. Activity device 142 can be any kind of device usable in any activities artificial intelligence model 126 may recommend. Accordingly, activity device 142 may comprise a therapeutic device, such as, for example, a percussive massage device, a pneumatic compression therapy device, a massage roller, a skin treatment device, a facial device, a wearable device, a guided breathing device, a guided meditation device, a heart rate modulating device, a temperature therapy device, a vibration therapy device, a cupping therapy device, a neuromuscular electrical stimulation device, a transcutaneous electrical nerve stimulation device, a massage chair, a therapeutic mat, a photobiomodulation device, sleep mask, eye massager, or any combination of the foregoing. In further embodiments, activity device 142 may comprise a monitoring device, such as an athletic activity monitoring device, a heart rate monitor, a step counter, or any other device capable of monitoring user activities, such as a smart watch, smart ring, smart earring, or the like. In further embodiments, activity device 142 may monitor a user’s health and/or sleep, including monitoring a user’s nutrition intake, caffeine intake, alcohol intake, and the like. In further embodiments, activity device 142 may monitor a user’s sleep activities, including monitoring sleep durations, types of sleep (light sleep, deep sleep, and REM sleep), time spent awake, time to fall asleep, and the like. In further embodiments, activity device 142 may comprise a smartphone or mobile device for image capture, video capture, and self-reported and/or monitored user inputs (e.g., for user information related to musculoskeletal health data, posture, movement, pain intensity levels, and the like).

Activity device 142 may communicate some or all of the user information to electronic computing device 130. For example, activity device 142 may communicate user information in the form of a user’s activity history to electronic computing device 130. Activity history can comprise, for example, recovery activities, athletic activities, and/or sleep activities performed by a user.

Communication between electronic computing device 130 and activity device 142 may therefore enable application 110 to automatically determine that a user has completed activities recommended by artificial intelligence model 126. Communication between electronic computing device 130 and activity device 142 may also automatically provide application 110 with user information in the form of activity history that artificial intelligence model 126 may use when determining its recommendations to the user. For example, where activity device 142 comprises a recovery device and a user uses activity device 142 for a recovery activity, activity device 142 may communicate user information in the form of records of that usage to electronic computing device 130. Similarly, where activity device 142 comprises a monitoring device and a user engages in athletic activity and/or sleep activity observable by activity device 142, activity device 142 may communicate user information in the form of records of the athletic activity and/or sleep activity to electronic computing device 130. In another example, where activity device 142 comprises a recovery device (such as a sleep mask or eye massager) and a user uses activity device 142 for a sleep activity, activity device 142 may communicate user information in the form of records of that usage to electronic computing device 130. With such records, application 110 may be able to automatically determine that the user has completed activities recommended by artificial intelligence model 126, and artificial intelligence model 126 may use such records when determining what activities to recommend to the user.

In some embodiments, electronic computing device 130 may be configured to communicate information related to activities recommended by artificial intelligence model 126 to activity device 142. In some embodiments, the information related to activities recommended by artificial intelligence model 126 may comprise operating parameters for activity device 142. Communication between electronic computing device 130 and activity device 142 may thereby create a convenient user experience. For example, if artificial intelligence model 126 recommends a specific percussive massage protocol, electronic computing device 130 may communicate appropriate percussive massage frequency, force, duration, or any combination of the foregoing to activity device 142. In another example, if artificial intelligence model 126 recommends a specific vibration protocol, electronic computing device 130 may communicate appropriate vibration patterns, vibration intensity, duration, or any combination of the foregoing to activity device 142. In yet another example, if artificial intelligence model 126 recommends a specific temperature therapy protocol, electronic computing device 130 may communicate appropriate temperature levels, duration, or any combination of the foregoing to activity device 142. In yet another example, if artificial intelligence model 126 recommends a specific light therapy protocol, electronic computing device 130 may communicate appropriate wavelength, fluence, power density, mode (continuous wave or pulsed light), duration, or any combination of the foregoing to activity device 142. The user may thereafter be able to activate the recommended protocol on activity device 142 without having to input the communicated parameters to activity device 142, thus reducing the time and effort needed to perform the activity recommended by artificial intelligence model 126.

In some embodiments, electronic computing device 130 may act as activity device 142. Thus, any of the functions described above as being achievable by communication between electronic computing device 130 and activity device 142 may be achieved by internal communication between different processes, applications, or components of electronic communication device. For example, in some embodiments, a percussive massage device may be configured to run application 110, and may therefore act as both electronic computing device 130 and a therapeutic activity device 142. In some such embodiments, application 110 may cause a motor of the percussive massage device to operate according to a recommended percussive massage therapy protocol, application 110 may be able to monitor operation of components of the percussive massage device to detect completion of percussive massage therapy, or both. In further embodiments, a user’s smart phone may run both application 110 and a running training application so that the smart phone may act as both electronic computing device and therapeutic activity device. The running training application may, for example, use location or step counting functions to determine where, how quickly, or how far a user has run, and may communicate some or all of that information to application 110. Similarly, application 110 may, for example, send activity parameters in the form of running distance, route, duration, speed, or any combination of the foregoing to the running training application, so that the running training application may guide the user to complete an activity recommended by artificial intelligence model 126.

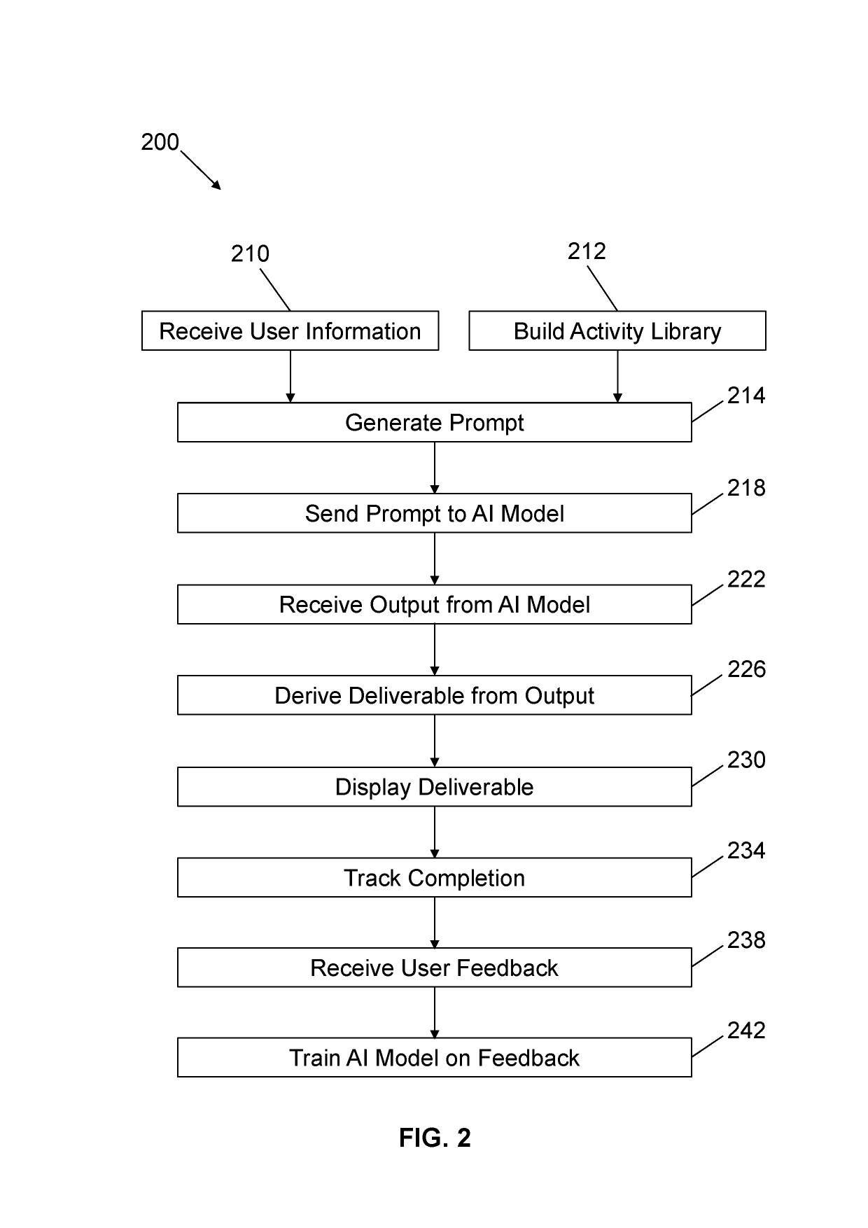

As shown in FIG. 2, an application-based user prompt-generation process 200 usable with system 100 comprises a user information receiving step 210 and a library building step 212. User information receiving step 210 comprises acquiring the user information discussed above and providing it to application 110. Library building step 212 comprises building activity library 146. In some embodiments, library building step 212 may also comprise building model prompt template library 148. User information receiving step 210 and library building step 212 may be involved in setting up application 110 for interacting with artificial intelligence model 126 and providing recommendations to a user. In embodiments wherein artificial intelligence model 126 is allowed to construct activities freely rather than selecting them from a predetermined activity library 146, library building step 212 may be omitted. In embodiments that include library building step 212, activity library 146 need not remain fixed after application-based user prompt generation process 200 progresses beyond the position of library building step 212 shown in FIG. 2.

Application-based user prompt generation process 200 further comprises application 110 interacting with artificial intelligence model 126 after application 110 is set up. Thus, application-based user prompt generation process 200 comprises a model prompt generating step 214 after user information receiving step 210 and, in some embodiments, library building step 212. Model prompt generating step 214 comprises model prompt builder 118 building a model prompt for artificial intelligence model 126. Application-based user prompt generation process 200 further comprises a model prompt sending step 218 following model prompt generating step 214. Model prompt sending step 218 comprises application 110 sending the model prompt generated during model prompt generating step 214 to artificial intelligence model 126. Application-based user prompt generation process 200 further comprises an output receiving step 222 following model prompt sending step 218. Output receiving step 222 comprises application 110 receiving output from artificial intelligence model 126 that artificial intelligence model 126 generates in response to the model prompt sent in model prompt sending step 218.

Application-based user prompt generation process 200 further comprises application 110 processing output received from artificial intelligence model 126 so that the output can be read by a user. Thus, application-based user prompt generation process 200 comprises a deliverable deriving step 226 following output receiving step 222. Deliverable deriving step 226 comprises deriving a deliverable from output received from artificial intelligence model 126 by processing the output with deliverable generator 122. Application-based user prompt generation process 200 also comprises a deliverable displaying step 230 following deliverable deriving step 226. Deliverable displaying step 230 comprises using user interface 114 to display the deliverable derived in deliverable deriving step 226.

Application-based user prompt generation process 200 may also comprise a completion tracking step 234 following deliverable displaying step 230. Completion tracking step 234 may comprise receiving confirmation a user has completed the activities recommended by artificial intelligence model 126 from user inputs, from information recorded by activity device 142, or both.

Application-based user prompt generation process 200 may optionally also comprise using user feedback to train artificial intelligence model 126. Thus, in some embodiments, application-based user prompt generation process 200 may comprise a feedback receiving step 238 following completion tracking step 234. Feedback receiving step 238 comprises receiving a user’s feedback on artificial intelligence model’s 126 recommendations. Feedback receiving step 238 may optionally comprise actively soliciting feedback from the user about a recommended activity having a most recently tracked completion. In further embodiments, feedback receiving step 238 may optionally comprise asking a user why a recommended activity was not completed.

Where a user has completed a user activity recommended by artificial intelligence model 126, feedback receiving step 238 may comprise receiving the user’s feedback concerning overall satisfaction with the recommended activity, the user’s enjoyment of the recommended activity, or the recommended activity’s success in contributing to specific objectives such as the user’s recovery from athletic activities, alleviating muscle soreness, achieving athletic goals, improving sleep, or reducing acute or chronic pain.

Application-based user prompt generation process 200 may comprise a model training step 242 following feedback receiving step 238. Model training step 242 may comprise sending feedback received during feedback receiving step 238 to artificial intelligence model 126 and training artificial intelligence model 126 on the feedback to improve artificial intelligence model’s 126 processes for generating recommendations based on user information.

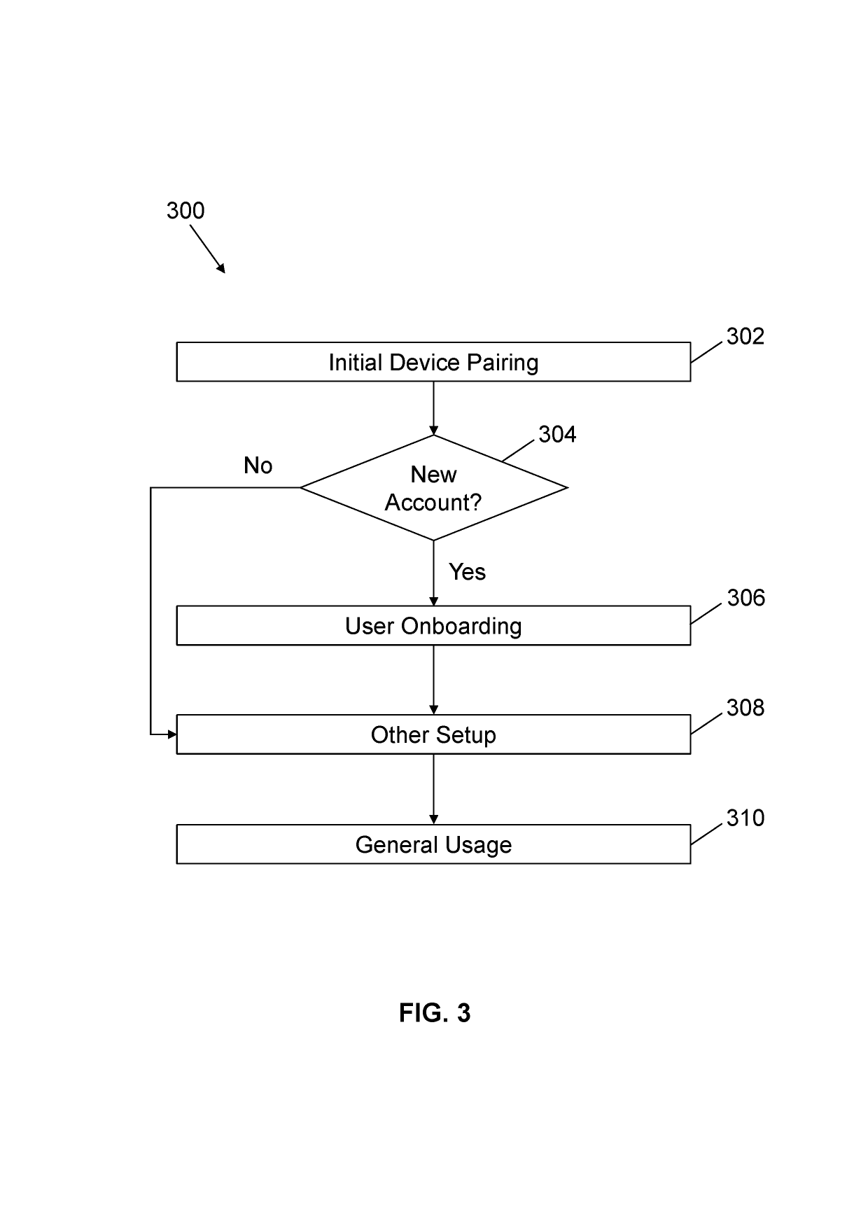

FIG. 3 shows an application setup workflow 300 for setting up application 110 to operate on electronic computing device 130. Application 110 may include one or more pages, screens, or popups dedicated to each step or decision in application setup workflow 300. Thus, each step and decision in application setup workflow 300 may correspond to at least one dedicated page, screen, or popup in application 110.

Application setup workflow 300 may comprise an initial device pairing step 302. Application setup workflow 300 can comprise a process for preparing application 110 for user. Initial device pairing step 302 may comprise pairing an activity device 142 with electronic computing device 130 so that activity device 142 may communicate with application 110. In the illustrated embodiment, application setup workflow 300 begins with initial device pairing step 302, but initial device pairing step 302 may occur later in other embodiments. In some embodiments, use of an activity device 142 may be a mandatory aspect of usage of system 100 and of application-based user prompt generation process 200. In other embodiments, use of an activity device 142 may be optional to usage of system 100 and of application-based user prompt generation process 200, and in such embodiments, initial device pairing step 302 may be moved later in application setup workflow 300 or omitted.

Application setup workflow 300 comprises a new account decision 304. In some embodiments, new account decision 304 may follow initial device pairing step 302. New account decision 304 comprises determining whether the user is setting up application 110 with a new account or signing into application 110 with an existing account.

If new account decision 304 results in a determination that yes, the user is setting up a new account, application setup workflow 300 proceeds to a user onboarding step 306. User onboarding step 306 may comprise receiving background user information that may be usable in a prompt for artificial intelligence model 126.

Application setup workflow 300 also comprises an other setup step 308. Other setup step 308 may comprise, for example, receiving user information usable for application-based user prompt generation process 200 but not stored with the user account, granting application 110 necessary permissions, or linking other processes and applications of electronic computing device 130 with application 110. For example, other setup step 308 may comprise providing a user an opportunity to link an account for a step counting application, a run tracking application, any other type of activity tracking application, a sleep tracking application, or a health or biometric monitoring application, with application 110 so that application 110 may receive data from the linked application as user information. If new account decision 304 results in a determination that no, the user is not setting up a new account, and is instead signing into application 110 with an existing account, application setup workflow 300 may bypass user onboarding step 306 by proceeding directly from new account decision 304 to other setup step 308. Application setup workflow 300 may also proceed from user onboarding step 306 to other setup step 308.

Following completion of other setup step 308, application setup workflow 300 may proceed to a general usage state 310 of application 110. General usage state 310 may be a state wherein application 110 provides an intended user experience and enables navigation between the various functions of application 110. Thus, general usage state 310 may enable user to update user information, request recommendations from artificial intelligence model 126, access deliverables derived from recommendations from artificial intelligence model 126, or replace, remove, or add paired activity devices 142.

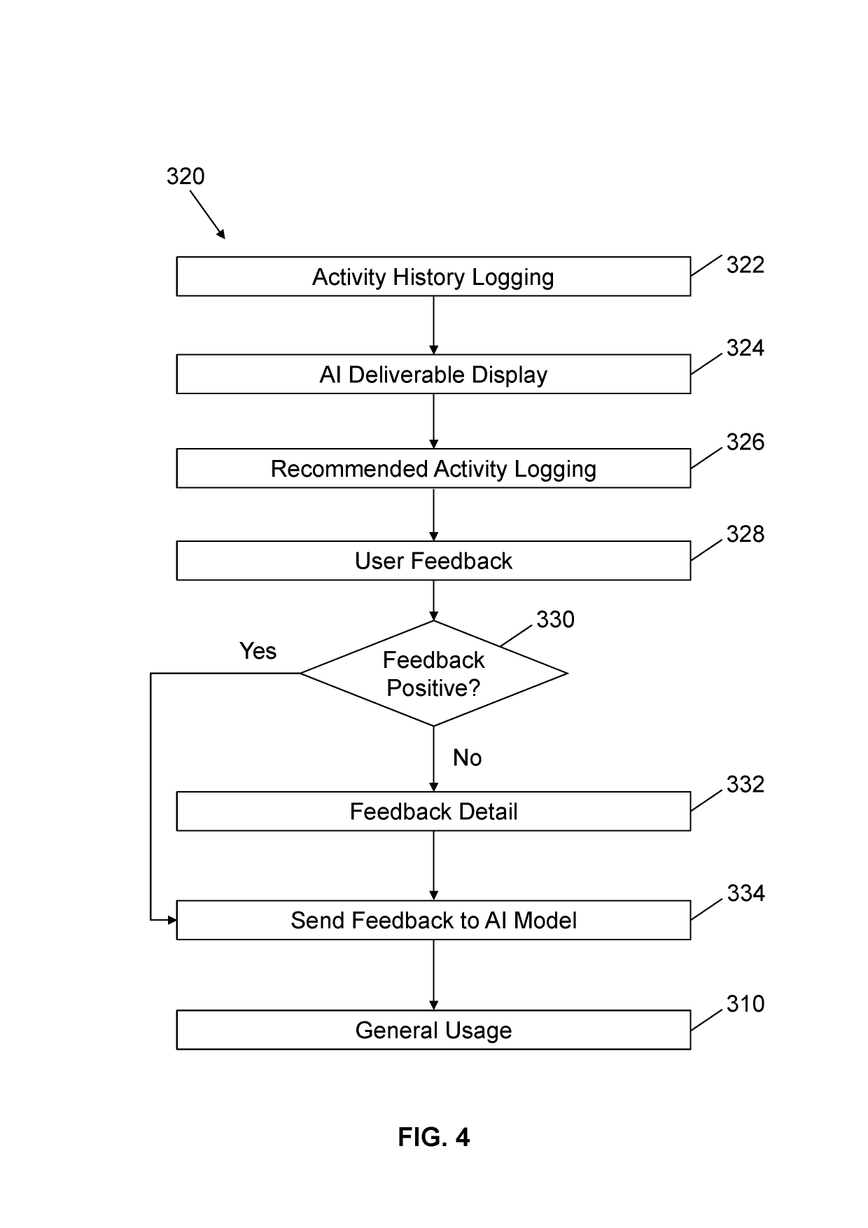

FIG. 4 shows an artificial intelligence workflow 320. Artificial intelligence workflow 320 may comprise user-facing aspects of a process within application 110 that relates to receiving user information usable to prompt or artificial intelligence model 126 and providing deliverables derived from artificial intelligence model’s 126 output to the user. Thus, application 110 may proceed through some or all steps within artificial intelligence workflow 320 within application-based user prompt generation process 200. Application 110 may include one or more pages, screens, or popups dedicated to each step or decision in artificial intelligence workflow 320. Thus, each step and decision in artificial intelligence workflow 320 may correspond to at least one dedicated page, screen, or popup in application 110.

Artificial intelligence workflow 320 comprises an activity history logging step 322. Activity history logging step 322 comprises a user logging any activities the user has completed that may be relevant to the recommendation the user seeks from artificial intelligence model 126. For example, during activity history logging step 322, the user may use application 110 to log recently completed workouts so that artificial intelligence model 126 may recommend appropriate recovery activities. In another example, during activity history logging step 322, the user may use application 110 to log recent sleep activities so that artificial intelligence model 126 may recommend appropriate sleep activities with activity device 142 to improve future sleep. The user may bypass activity history logging step 322 if application 110 has received relevant activities from activity device 142 or from other applications operating on electronic computing device 130.

Artificial intelligence workflow 320 further comprises a deliverable displaying step 324 following activity history longing step 322. Deliverable displaying step 324 comprises displaying the deliverable derived from the output of artificial intelligence model 126 by deliverable generator 122 as described above in connection with deliverable deriving step 226 and deliverable displaying step 230 of application-based user prompt generation process 200. Thus, deliverable displaying step 324 comprises displaying artificial intelligence model’s 126 recommendations in a format readable by the user.

Artificial intelligence workflow 320 further comprises a recommended activity logging step 326 following deliverable displaying step 324. Recommended activity logging step 326 comprises a user logging completion of activities recommended by artificial intelligence model 126. Recommended activity logging step 326 may therefore overlap with completion tracking step 234 of application-based user prompt generation process 200.

Artificial intelligence workflow 320 may further comprise a user feedback step 328 following recommended activity logging step 326. User feedback step 328 may comprise prompting a user to provide feedback on activities recommended by artificial intelligence model 126 as described above with regard to feedback receiving step 238 of application-based user prompt generation process 200. Artificial intelligence workflow 320 may further comprise a feedback positivity decision 330 following user feedback step 328. Feedback positivity decision 330 may comprise determining whether the user feedback is positive. For example, the user feedback options provided at user feedback step 328 may be a strictly binary or numerical assessment of positivity, such as thumbs up and thumbs down indicators or a start scale rating. Only a predetermined subset among the options provided during user feedback step 328 may be considered positive. If feedback positivity decision 330 results in a determination that no, the user feedback is not positive, artificial intelligence workflow 320 may proceed to a feedback detail step 332 to solicit reasons for a user’s dissatisfaction with artificial intelligence model’s 126 recommendations.

Artificial intelligence workflow 320 further comprises a feedback sending step 334, in which feedback received during user feedback step 328 is sent to artificial intelligence model 126 for the purpose of training artificial intelligence model 126. Feedback sending step 334 may also comprise sending any additional feedback details received during feedback detail step 332 to artificial intelligence model 126 for the purpose of training artificial intelligence model 126.

In embodiments including feedback detail step 332, artificial intelligence workflow 320 may proceed from feedback detail step 332 to feedback sending step 334. In some embodiments, if feedback positivity decision 330 results in a determination that yes, the user feedback received in user feedback step 328 is positive, artificial intelligence workflow 320 may bypass feedback detail step 332 and proceed from feedback positivity decision 330 to feedback sending step 334. Feedback detail step 332 may be bypassed where the initial user feedback is positive because it may be relatively safe to assume that positive feedback indicates that artificial intelligence model’s 126 recommendations have succeeded in their objectives, meaning it may be more efficient to proceed without asking the user why the initial feedback was positive. However, a user may become dissatisfied as a result of a recommendation’s failure at any criteria, even including criteria that artificial intelligence model 126 or the designers of application 110 may not have considered. For that reason, the additional detail solicited in feedback detail step 332 can contribute significantly to the value of negative feedback for the purpose of training artificial intelligence model 126. However, in further embodiments, feedback positivity decision 330 may be omitted such that artificial intelligence workflow 320 may always proceed from user feedback step 328 to feedback detail step 332 before proceeding to feedback sending step 334. In still further embodiments, feedback positivity decision 330 and feedback detail step 332 may both be omitted so that artificial intelligence workflow 320 may proceed from user feedback step 328 to feedback sending step 334 without soliciting additional detail regardless of the positivity of the feedback.

User feedback step 328, feedback positivity decision 330, and feedback detail step 332 may overlap with feedback receiving step 238 of application-based user prompt generation process 200.

After feedback detail step 332, artificial intelligence workflow 320 may return application 110 to general usage state 310.

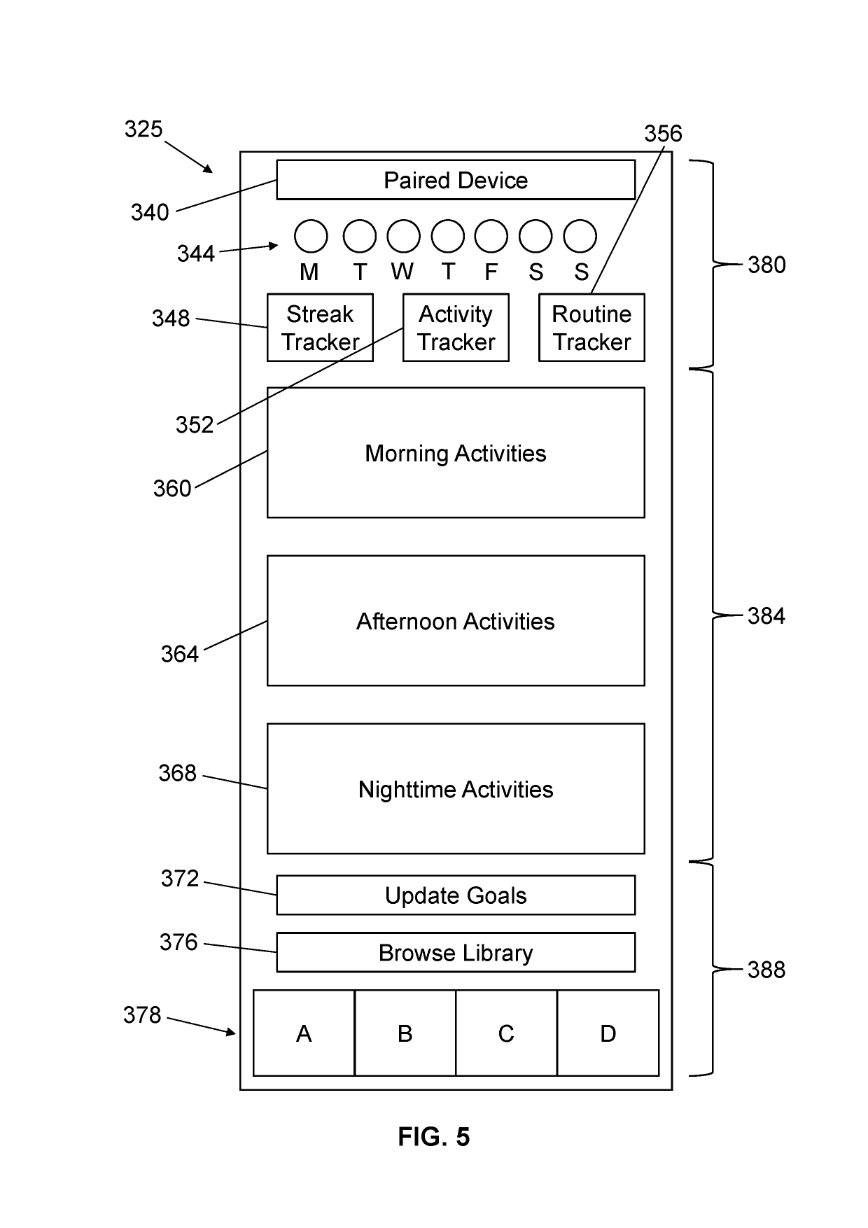

FIG. 5 illustrates an example of a deliverable display screen 325. Deliverable display screen 325 may be a state of user interface 114 of application 110. Application 110 may cause user electronic computing device 130 to show deliverable display screen 325 at deliverable displaying step 230 of application-based user prompt generation process 200 and when application 110 is at deliverable displaying step 324 of artificial intelligence workflow 320.

Deliverable display screen 325 comprises a deliverable area 384 in which part or all of the deliverable derived from output from artificial intelligence model 126 is presented. Deliverable area 384 of the illustrated example comprises a morning deliverable portion 360, an afternoon deliverable portion 364, and a nighttime deliverable portion 368, ordered from earliest to latest. Morning deliverable portion 360 comprises activities artificial intelligence model 126 recommended to be performed during the morning, afternoon deliverable portion 364 comprises activities artificial intelligence model 126 recommended to be performed during the afternoon, and nighttime deliverable portion 368 comprises activities artificial intelligence model 126 recommended to be performed at night. Thus, deliverable display screen 325 of the illustrated example presents activities recommended by artificial intelligence model 126 in groups according to the time of day at which artificial intelligence model 126 recommends performing them.

In some embodiments, deliverable area 384 may be a scrollable area. That is, electronic computing device 130 may display only a portion of deliverable area 384, with deliverable area 384 extending beyond the displayed portion, and a user may scroll across deliverable area 384 to change which portion of deliverable area 384 is displayed. The deliverable in deliverable area 384 may therefore contain a list of recommended activities too long to be displayed in its entirety within the available area of deliverable display screen 325, with the user being able to access all recommended activities by scrolling. In some embodiments, deliverable area 384 may be scrollable vertically, but not horizontally. In further embodiments, deliverable area 384 may be scrollable horizontally, but not vertically. In further embodiments, deliverable area 384 may be scrollable vertically and horizontally.

In some embodiments, deliverable display screen 325 may comprise a header 380 fixed above deliverable area 384 or a footer 388 fixed below deliverable area 384. In the illustrated embodiment, deliverable display screen 325 comprises both a header 380 fixed above deliverable area 384 and a footer 388 fixed below deliverable area 384. In this instance, header 380 being fixed above deliverable area 384 means that header 380 may remain visible above deliverable area 384 even as a user scrolls across different portions of the deliverable within deliverable area 384. Similarly, in this instance, footer 388 being fixed below deliverable area 384 means that footer 388 may remain visible below deliverable area 384 even as a user scrolls across different portions of the deliverable within deliverable area 384.

Header 380 may comprise one or more indicators, buttons, or selectable elements. For example, header 380 of the illustrated example comprises a paired device indicator 340. Paired device indicator 340 may indicate a selected activity device 142 that is paired to electronic computing device 130. Paired device indicator 340 may comprise a dropdown menu so that a user may select one among multiple activity devices 142 paired to electronic computing device 130 by interacting with paired device indicator 340. In some embodiments, deliverable area 384 may update in response to user inputs to display only recommended activities that involve an activity device 142 indicated to be currently selected by paired device indicator 340.

Header 380 of the illustrated embodiment comprises a weekday tracker 344. Weekday tracker 344 comprises a portion dedicated to each day of the week. In some embodiments, each portion of weekday tracker 344 may change in appearance to indicate different degrees of completion of recommended activities scheduled for the respective day. In further embodiments, each portion of weekday tracker 344 may be selectable by user to cause deliverable area 384 to display only activities recommended to be completed on the day corresponding to the portion of weekday tracker 344 selected by the user.

Header 380 of the illustrated embodiment comprises additional trackers. In particular, header 380 comprises a streak tracker 348, an activity tracker 352, and a routine tracker 356. Streak tracker 348 may indicate a number of consecutive days a user has completed activities recommended by artificial intelligence model 126. In other embodiments, streak tracker 348 may indicate a number of consecutive days a user has used activity device 142. Activity tracker 352 may indicate how many activities recommended by artificial intelligence model 126 a user has completed out of a total number of activities recommended by artificial intelligence model 126 within a predetermined timeframe. In further embodiments, activity tracker 352 may indicate a number of activities reported as completed by synced applications, such as a running training application or a step counting application. Routine tracker 356 may indicate how many recommended routines from activity library 146 a user has completed. Streak tracker 348 may indicate whether a user has used their activity device 142 at all for that calendar day which may include, any activities recommended by the artificial intelligence model 126 through the application 110 and any offline activity device 142 usage that has been synced that day (e.g., usage of the activity device 142 without using application 110). Activity tracker 352 may show the number of activities synced and completed from third-party applications, e.g., Garmin, Apple Health, Strava, Google Fit, and the like. Routine tracker 356 may indicate the number of any Theragun routines a user completes in the entire app including any offline device presets (not just general usage) that is synced that day. Routine tracker 356 may track this all for the week and show the difference compared to last week next to each number, e.g. up 2, down 1, etc.

Footer 388 of the illustrated embodiment comprises a goal button 372. When selected, goal button 372 may cause user interface 114 to display the user’s previously selected goals, and may further allow the user to update the selected goals by deselecting previously selected goals or selecting new goals. The selected goals may be within the user information provided to artificial intelligence model 126 for the artificial intelligence model 126 to consider as inputs when developing recommendations for the user.

Footer 388 of the illustrated embodiment comprises a library button 376. Library button 376 may cause user interface 114 to display a screen wherein a user may browse a library of activities, such as activity library 146.

Footer 388 of the illustrated embodiment comprises a navigation bar 378. Navigation bar 378 may comprise a group of buttons for navigating to different pages or functions of application 110. A group of four buttons labeled A, B, C, and D is shown by way of example in FIG. 5, but navigation bar 378 may comprise any number of buttons with any assortment of labels appropriate to a given implementation of application 110.

The particular elements and arrangement thereof in deliverable display screen 325 as illustrated in FIG. 5 represent an example of how deliverable display screen 325 may be embodied, but deliverable display screen 325 may vary significantly in other embodiments. For example, either or both of header 380 and footer 388 may be omitted. Any elements shown in header 380 and footer 388 of FIG. 5 may be relocated to the other of header 380 or footer 388 or may be relocated into a scrollable area that includes deliverable area 384. In further embodiments, deliverable area 384 may not be divided into distinct parts of the day, or may not be chronologically arranged at all.

It is to be appreciated that the Detailed Description section, and not the Summary and Abstract sections, is intended to be used to interpret the claims. The Summary and Abstract sections may set forth one or more but not all exemplary embodiments of the present disclosure as contemplated by the inventor(s), and thus, are not intended to limit the present disclosure and the appended claims in any way.

Embodiments of the present disclosure have been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the disclosure that others can, by applying knowledge within the skill of the art, readily modify and/or adapt for various applications such specific embodiments, without undue experimentation, without departing from the general concept of the present disclosure. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary embodiments, but should be defined only in accordance with the following claims and their equivalents.

What is claimed is:

1. An electronic computing device configured to:
receive user information, wherein the user information comprises usage history of a therapeutic device received through electronic communication from the therapeutic device;
generate a model prompt comprising:
the user information,
instructions to select a user activity based on the user information,
instructions to output a recommendation of the user activity, and
instructions to output the recommendation in a standardized data format;
send the model prompt to an artificial intelligence model;
receive an output comprising the recommendation from the artificial intelligence model;
derive a deliverable from the output by processing the output in a routine configured to generate deliverables from information in the standardized data format; and
display the deliverable.

2. The electronic computing device of claim 1, wherein the instructions to select the user activity comprise instructions to select the user activity from a predefined group of activities.

3. The electronic computing device of claim 1, further configured to display the deliverable within a graphical user interface, wherein the deliverable is derived from the output.

4. The electronic computing device of claim 1, wherein the routine is configured to arrange information in the standardized data format in a graphical display format.

5. The electronic computing device of claim 1, further configured to receive user feedback on the recommendation and send the feedback to the artificial intelligence model.

6. The electronic computing device of claim 1, wherein the user activity comprises a therapeutic activity.

7. The electronic computing device of claim 1, wherein the user information comprises a user characteristic.

8. The electronic computing device of claim 1, wherein the user information comprises activity history.

9. The electronic computing device of claim 8, wherein the activity history comprises information transmitted from an activity monitoring device.

10. The electronic computing device of claim 1, wherein the user information comprises a user goal.

11. The electronic computing device of claim 1, further configured to detect completion of the user activity from data transmitted from the therapeutic device.

12. An electronic computing device configured to:
receive user information comprising:
user goals, and
activity history transmitted from an activity monitoring device;
prompt an artificial intelligence model to select a recovery activity based on the user information and to output a recommendation comprising the recovery activity; and
display a deliverable derived from the recommendation.

13. The electronic computing device of claim 12, wherein the recommendation is one among a plurality of recommendations comprised by the deliverable, and each recommendation among the plurality of recommendations comprises a time aspect, and wherein the electronic computing device is configured to display the deliverable in a format that lists the plurality of recommendations in chronological order according to the time aspect of each recommendation.

14. The electronic computing device of claim 12, further configured to detect completion of the recovery activity from data transmitted from a therapeutic device.

15. A method of generating prompts for a user, the method comprising:
requesting user information from a user;
generating a model prompt comprising:
the user information,
instructions to select a user activity based on the user information,
instructions to output a recommendation of the user activity, and
instructions to output the recommendation in a standardized data format;
sending the model prompt to an artificial intelligence model;
generating a deliverable by processing an output comprising the recommendation in a routine configured to generate deliverables from information in the standardized data format; and
providing the deliverable to the user.

16. The method of claim 15, further comprising:
receiving user feedback on the recommendation; and
training the artificial intelligence model on the user feedback.

17. The method of claim 15, further comprising:
tracking completions of recommended activities by the user, wherein the recommended activities comprise the user activity; and
providing feedback to the user based on the completions.

18. The method of claim 17, wherein the user activity comprises usage of a therapeutic device, and tracking completions of recommended activities comprises receiving data transmitted by the therapeutic device.

19. The method of claim 15, further comprising building a predetermined group of user activities, wherein the model prompt comprises instructions to select the user activity from the predetermined group of user activities.

20. The method of claim 15, wherein receiving the user data comprises receiving data transmitted from an activity monitoring device.
